(19) United States
(12) Patent Application Publication
(10) Pub. No.: US 2023/0346605 A1
(43) Pub. Date: Nov. 2, 2023

(54) DEVICES, SYSTEMS AND METHODS FOR TREATING AND PREVENTING VENOUS INSUFFICIENCY, THROMBOSIS, ORTHOSTATIC INTOLERANCE, AND IMPAIRED LYMPHATIC DRAINAGE

(71) Applicant: ORBITAL BIODESIGN, LLC, Boulder, CO (US)

(72) Inventors: Danielle J. CARROLL, Boulder, CO (US); Jana R. MONTGOMERY, Nederland, CO (US)

(21) Appl. No.: 18/118,474

(22) Filed: Mar. 7, 2023

Related U.S. Application Data
(63) Continuation-in-part of application No. 17/571,230, filed on Jan. 7, 2022.
(60) Provisional application No. 63/317,127, filed on Mar. 7, 2022.
(60) Provisional application No. 63/134,664, filed on Jan. 7, 2021.

(51) Int. Cl. A61F 13/08

(52) U.S. Cl. CPC A61F 13/085

(57) ABSTRACT

An apparatus for treating venous insufficiency is disclosed. The apparatus includes a plurality of actuator assemblies receivable on a limb of a user. The actuator assemblies include: a flexible element including a body portion and two opposing end portions. The body portion is configured to at least partly extend around an outer periphery of the limb. An actuator is coupled to the opposing end portions and configured to selectively reduce a distance between the two end portions to compress the limb.

### CROSS-REFERENCE TO RELATED APPLICATIONS

This application is a continuation-in-part of U.S. patent application Ser. No. 17/571,230, filed 7 Jan. 2022, entitled “DEVICES, SYSTEMS AND METHODS FOR TREATING AND PREVENTING VENOUS INSUFFICIENCY, THROMBOSIS, ORTHOSTATIC INTOLERANCE, AND IMPAIRED LYMPHATIC DRAINAGE,” which claims the benefit of priority under 35 U.S.C. § 119(e) of U.S. provisional patent application No. 63/134,664, filed 7 Jan. 2021, entitled “DEVICES, SYSTEMS AND METHODS FOR TREATING AND PREVENTING VENOUS INSUFFICIENCY,” and this application claims the benefit of priority pursuant to 35 U.S.C. § 119(e) of U.S. provisional patent application No. 63/317,127, filed 7 Mar. 2022, entitled “DEVICES, SYSTEMS AND METHODS FOR TREATING AND PREVENTING VENOUS INSUFFICIENCY, THROMBOSIS, ORTHOSTATIC INTOLERANCE, AND IMPAIRED LYMPHATIC DRAINAGE,” all of which are hereby incorporated by reference herein in their entirety for all purposes.

### BACKGROUND

The venous network in humans is characteristically a low-pressure system, which necessitates the presence of staggered valves in order to prevent backflow as blood from the lower legs is pumped against gravity and back toward the heart. The calf (gastrocnemius) muscle assists with the pumping action during walking, thereby facilitating venous return, but individuals in career fields that involve prolonged standing are at increased risk of a condition known as venous insufficiency, in which the peripheral vascular system becomes overwhelmed by stagnant blood due to inactivation of the calf pump. Here, superficial veins dilate in order to accommodate the venous congestion that results, which can ultimately lead to painful and unsightly varicose veins.

Currently, compression socks and sleeves are available to help combat this issue, but with limited effectiveness. Moreover, the currently available compression socks, sleeves and related apparatuses require frequent replacement due to limited retention of elasticity over time.

What is needed are improved apparatuses, wearables and/or medical devices that effectively prevent, mitigate and/or treat venous insufficiency and related/resultant conditions. Such is provided by the presently disclosed subject matter.

### BRIEF SUMMARY

In one embodiment, an apparatus for treating venous deficiency is disclosed. The apparatus includes a plurality of actuator assemblies receivable on a limb of a user. Each of the plurality of actuator assemblies include a flexible element with a body portion and two opposing end portions. The body portion is configured to at least partly extend around an outer periphery of the limb. An actuator is coupled to the opposing end portions and configured to selectively tension the body portion by reducing a distance between the two end portions to compress the limb.

Optionally, in some embodiments, the plurality of actuator assemblies includes a first actuator assembly and a second actuator assembly, and the selective tensioning of the first actuator assembly temporally spaced from the selective tensioning of the second actuator assembly such that the apparatus induces a peristaltic pressure wave in the limb.

Optionally, in some embodiments, the actuator includes a rotary actuator.

Optionally, in some embodiments, the rotary actuator includes at least one of a stepper motor or a servo motor.

Optionally, in some embodiments, the apparatus further includes a tensioner rotationally coupled to the rotary actuator and to the opposing end portions, such that a rotation of the rotary actuator causes the end portions of the flexible element to at least partially wrap around the tensioner to reduce the distance between the two end portions.

Optionally, in some embodiments, the apparatus further includes a sleeve wearable on the limb of the user, wherein the plurality of actuator assemblies is coupled to the sleeve.

Optionally, in some embodiments, the apparatus further includes a closure mechanism coupled to the sleeve and operative to selectively open the sleeve for ease of placement on the limb.

Optionally, in some embodiments, the closure mechanism includes at least one of a zipper, a button, a clasp, a hook and loop fastener, or hook and pile fastener.

Optionally, in some embodiments, the apparatus further includes a sensor configured to measure one or more of a tension in the flexible element or a compression in the limb.

Optionally, in some embodiments, the apparatus tensions the flexible element until a desired level of compression in the limb is applied by the flexible element, as measured by the sensor.

Optionally, in some embodiments, the desired level of compression is in the range of 30 mmHg to 200 mmHg.

Optionally, in some embodiments, the selective tensioning of the first actuator assembly and the selective tensioning of the second actuator assembly at least partially overlap temporally.

Optionally, in some embodiments, the sensor includes one or more of a force sensing resistor, a strain gauge, or a Wheatstone bridge.

Optionally, in some embodiments, the sensor is coupled to the flexible element.

Optionally, in some embodiments, the sensor is coupled to the flexible element by one or more of stitching or adhesive.

Optionally, in some embodiments, the flexible element includes a plurality of layers, and the sensor is disposed between at least two layers of the plurality of layers.

Optionally, in some embodiments, order of the selective tensioning of the first actuator and the selective tensioning of the second actuator is such that the peristaltic pressure wave is a cranial or proximal pressure wave.

Optionally, in some embodiments, an order of the selective tensioning of the first actuator and the selective tensioning of the second actuator is such that the peristaltic pressure wave is a caudal or distal pressure wave.

Optionally, in some embodiments, the sleeve includes a woven or non-woven fabric.

Optionally, in some embodiments, the sleeve is conformable to the limb.

Optionally, in some embodiments, the sleeve has a circumference of about 10 cm to about 60 cm.

Optionally, in some embodiments, the sleeve has a thickness of about 5 mm to about 1.5 cm.

Optionally, in some embodiments, the peristaltic pressure wave has a frequency of about 20 to 60 waves per minute.

Optionally, in some embodiments, the apparatus induces a second pressure wave in the limb at the same time and different location as the pressure wave.

A method of treating venous deficiency is disclosed. The method includes applying a wearable device to a limb of a user. The wearable device includes a plurality of actuator assemblies receivable on a limb of a user. The plurality of actuator assemblies include a flexible element including a body portion and two opposing end portions. The body portion is configured to at least partly extend around an outer periphery of the limb, and an actuator coupled to the opposing end portions and configured to selectively tension the body portion by reducing a distance between the two end portions to compress the limb. Selective tensioning of adjacent actuator assemblies induces a peristaltic pressure wave in the limb.

Optionally, in some embodiments, the peristaltic pressure wave has a magnitude of between about 30 mmHg and 200 mmHg.

Optionally, in some embodiments, the peristaltic pressure wave has a frequency of about 20 to 60 waves per minute.

### BRIEF DESCRIPTION OF THE SEVERAL VIEWS OF THE DRAWINGS

FIG. 1 is a simplified view of wearable devices in place on a user's a lower leg.

FIG. 2A is an example of a peristaltic pressure wave induced by a wearable device of FIG. 1.

FIG. 2B is an example of a peristaltic pressure wave induced by a wearable device of FIG. 1.

FIG. 3A is a simplified view of an actuator assembly for use with the wearable device of FIG. 1 in a first configuration.

FIG. 3B is a view of the actuator assembly of FIG. 3A in a second configuration.

FIG. 4A is an anterior elevation view of an example of the wearable device of FIG. 1.

FIG. 4B is a lateral elevation view of the wearable device of FIG. 4A.

FIG. 4C is a posterior elevation view of the wearable device of FIG. 4A.

FIG. 5 is an example of a timing diagram of for a pressure wave induced by a wearable device of the present disclosure.

FIG. 6 is a simplified block diagram of components of the wearable device.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements. This application is a continuation-in-part of U.S. patent application Ser. No. 17/571,230, filed 7 Jan. 2022, which claims the benefit of priority of U.S. provisional patent application No. 63/134,664, filed 7 Jan. 2021, both of which are incorporated by reference herein in their entirety. This application also claims the benefit of priority of U.S. provisional patent application No. 63/317,127, filed 7 Mar. 2022, which is incorporated by reference herein in its entirety.

In humans, as well as other warm-blooded and mammalian species, the venous system functions at a characteristically low pressure. The venous network returns blood from distal structures, to include extremities (e.g. feet/legs and hands/arms), to the cardiopulmonary vasculature, to then reenter the arterial system. Due to the nature of this low-pressure system, the presence of staggered, one-way valves is necessary in order to prevent backflow as blood from extremities and limbs, and particularly the lower legs when in a standing position, is pumped against gravity and back toward the heart. At least in humans, the calf (gastrocnemius) muscle in the leg assists with the pumping action during walking and other movements of the legs, thereby facilitating and/or assisting in venous return. However, subjects that are sedentary for extended periods of time, and particularly standing for prolonged periods, e.g., individuals in career fields that involve prolonged standing, are at increased risk of a condition known as venous insufficiency.

Venous insufficiency occurs in subjects when the peripheral vascular system becomes overwhelmed by stagnant blood due to inactivation of the calf pump, i.e., during prolonged standing or other inactivity resulting in reduced blood flow from the extremities. In such instances, superficial veins dilate in order to accommodate the venous congestion that results, which can ultimately lead to painful and unsightly varicose veins.

The terminology used herein is for the purpose of describing particular embodiments and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”.

Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

In some embodiments, provided herein are apparatuses and/or devices for treating and/or preventing venous insufficiency, wherein the apparatuses and/or devices are configured to be worn by or applied to a subject, wherein the apparatuses and/or devices are configured to apply a peristaltic movement and/or peristaltic pressure to an affected region or area of the subject, wherein the apparatuses and/or devices comprise a soft robotic system capable of producing a peristaltic movement and/or peristaltic pressure within the apparatuses and/or devices. In some embodiments, peristaltic movement can be cranial/proximal for some applications (e.g., terrestrial/venous/lymphatic), and caudal/distal for other applications (e.g., spaceflight use). In some aspects, the soft robotic system capable of producing a peristaltic movement and/or peristaltic pressure within the apparatus and/or device comprises one or more synthetic muscle fibers, optionally wherein the one or more synthetic muscle fibers are configured to be activated concentrically by electrical impulse to mimic natural peristaltic movement.

In some aspects, the apparatuses and/or devices comprise a sleeve or foldable sheet of material, wherein the sleeve or foldable sheet of material comprises the soft robotic system capable of producing a peristaltic movement and/or peristaltic pressure within the apparatuses and/or devices. In some aspects, the sleeve can include an inner layer and an outer layer, wherein the inner layer comprises the soft robotic system, wherein the outer layer comprises a fabric material, wherein the sleeve is configured to be wearable over an appendage or limb of a subject. The soft robotic system of the inner layer can in some aspects comprise synthetic muscle fibers configured to be activated concentrically by electrical impulse to cause a natural peristaltic movement in an axial direction of the sleeve.

In some aspects, the inner layer and outer layer are separable, optionally wherein the inner layer and outer layer are attached and/or integrated. The inner layer can optionally comprise a silicone material, wherein the outer layer can optionally comprise a cotton or neoprene material. The sleeve can comprise any dimension suitable for application to a limb of a subject, optionally wherein the sleeve has a circumference of about 10 cm to about 60 cm (or about 10 cm, 20 cm, 30 cm, 40 cm, 50 cm or 60 cm) optionally wherein the sleeve has a length of about 10 cm to about 60 cm (or about 10 cm, 20 cm, 30 cm, 40 cm, 50 cm or 60 cm), optionally wherein the sleeve has a thickness of about 5 mm to about 1.5 cm (or about 5 mm, 6 mm, 7 mm, 8 mm, 9 mm, 1 cm, 1.1 cm, 1.2 cm, 1.3 cm, 1.4 cm or 1.5 cm). In some embodiments, the apparatus and/or device is configured to be wearable by a subject, optionally wherein the apparatus and/or device is configured to be placed on and/or over a limb, a leg or an arm of a subject, or otherwise applied to an affected area of the subject.

In some embodiments, apparatuses and/or devices are configured to apply a peristaltic movement or peristaltic pressure to an affected region or area of the subject, wherein a force applied to the affected region or area ranges from about 30 millimeters of mercury (mmHg) to about 200 mmHg. Additionally, in some aspects, the peristaltic movement or peristaltic pressure is applied at a frequency of about 20 to about 60 contractions per minute.

In some aspects, the apparatuses and/or devices further comprise a power source, optionally wherein the power source is a battery, optionally wherein the battery is rechargeable.

The apparatuses and/or devices can further comprise a computer, optionally wherein one or more functions of the apparatus and/or device are controlled by a processor of the computer, further comprising a computer readable medium having stored thereon computer executable instructions executable by the processor.

Provided herein are methods of treating and/or preventing venous insufficiency, the methods comprising applying the apparatuses and/or devices to a subject in need of treatment, wherein the subject is a human subject, optionally wherein the subject is susceptible to and/or is suffering from lower extremity varicose veins and/or edema. In such methods the apparatuses and/or devices are applied to one or more limbs or extremities of the subject, wherein the apparatuses and/or devices are controlled to apply peristaltic movement and/or peristaltic pressure to an affected region or area of the subject.

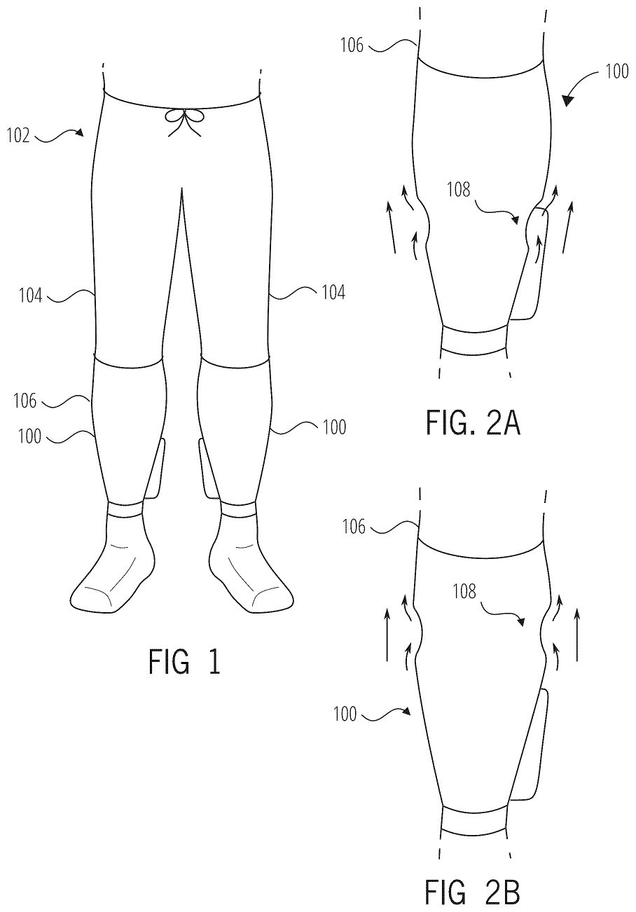

Provided herein are wearable apparatuses and/or devices configured to be placed on and/or over a limb, e.g. leg, arm, etc., of a subject, or otherwise applied to an affected area of a subject, where the apparatus and/or device is configured to apply a peristaltic movement or peristaltic pressure to an affected region. In some embodiments, such apparatuses and/or devices can comprise a sleeve or foldable sheet of synthetic muscle fibers that can be activated concentrically by electrical impulse to mimic natural peristaltic movement. See, e.g., FIGS. 1, 2A, and 2B.

The disclosed apparatuses and/or devices can comprise synthetic muscle or synthetic muscle fibers, including for example hydraulically amplified electrostatic actuators or other soft robotics that couple electrostatic and hydraulic forces to achieve diverse modes of actuation, to facilitate a natural peristaltic movement or pressure within the apparatus or device. Such synthetic muscle can be arranged within or integrated throughout the apparatus, including for example in a tube or sleeve structure that when activated concentrically via electrical impulse produces or mimics a natural peristaltic movement. In some embodiments, the disclose apparatuses and/or devices can include multiple layers of material/components, such as for example an inner layer (or inner sleeve or sheath) of synthetic muscle fibers surrounded by an outer layer (or outer sleeve or sheath) of material to support and/or maintain the orientation of the inner layer. Alternatively, and/or in addition, in some embodiments the synthetic muscle fibers can be integrated into and/or amongst other fibers or materials to form a composite layer that provides a sheet or sleeve of material with actuatable soft robotic capability to produce a peristaltic pressure. In some aspects, such materials, including the inner sleeve, outer sleeve, composite, etc., can include, but are not limited to cotton, polyester, neoprene, and blends thereof.

Such an apparatus and/or device, at least in applications for the leg, lower leg and/or calf area of the leg, can help to facilitate venous return by applying sequential external forces that propel blood from the lower leg back to the heart (FIGS. 1 and 2A/B). This alleviates gravity-induced pressure and can both prevent and treat venous insufficiency.

In some embodiments, the disclosed apparatus and/or device can be designed to be wearable by the user such that the user's mobility and/or day-today activities are not significantly affected by the device. In some aspects, the device and/or apparatus can be powered by a small, USB-rechargeable battery, and can be fully Bluetooth-capable and operable through a mobile application. To facilitate ease of use the device can be operable without being plugged into an electrical outlet, although in some aspects it can be configured to be used while plugged into an outlet. Such wearable designs can also be configured to be slim-fit such that they can be worn under clothing and outer wear.

The disclosed apparatuses and/or devices can be configured to apply sufficient pressure to modulate venous and lymphatic flow, while also being comfortable to the user. They can be configured to be easy to apply and remove, e.g., through the use of elastic and stretchable materials, and/or through the use of various fasteners, e.g., hook and loop or hook and pile closures, snaps, buttons, zippers and the like. Such apparatuses and/or devices can be designed to be reasonably quiet, i.e., low decibels, such that use of the device does not disturb the user or others nearby. Moreover, it can be Bluetooth-enabled and can be controlled by a device and/or mobile application.

In some embodiments, and by way of example, the disclosed apparatuses and/or devices can include one or more of the following specifications:

Materials
Silicone and other proprietary compounds
Covered in a thin, removable cotton sleeve

Dimensions
Circumference: 40 cm at its widest point
Length: 32 cm
Thickness: 2 mm (1.5 cm for small charging pack)

Pumping Action
Force Applied: 30 mmHg to 200 mmHg (controllable within app)
Frequency: 20 to 60 contractions per minute (controllable within app)

Charging Mechanism
USB-rechargeable battery

Additional specifications and examples, including materials, dimensions, and the like, are provided herein.

The subject matter disclosed herein can be implemented in software in combination with hardware and/or firmware. For example, the subject matter described herein can be implemented in software executed by a processor. In one exemplary implementation, the subject matter described herein can be implemented using a computer readable medium having stored thereon computer executable instructions that when executed by a processor of a computer control the computer to perform steps. Exemplary computer readable mediums suitable for implementing the subject matter described herein include non-transitory devices, such as disk memory devices, chip memory devices, programmable logic devices, and application-specific integrated circuits. In addition, a computer readable medium that implements the subject matter described herein can be located on a single device or computing platform or can be distributed across multiple devices or computing platforms.

With reference to FIG. 1, an example of a wearable device 100 is shown worn by a user 102. A wearable device 100 is shown on each of the user's 102 limbs 106. While the wearable device 100 shown for example may be adapted to be worn on the limbs 106, other embodiments, of wearable device 100 may be adapted to be worn on other extremities 104, such as a thigh, or upper or lower arm. The dimensions (e.g., circumference, length, thickness) of the wearable device 100 may be adapted for use different limbs and/or different parts of limbs. In one example, a wearable device adapted for use on an upper arm may have a length of about 20 cm and/or circumference of about 25 cm to 30 cm. In another example, a wearable device adapted for use on a lower arm may have a length of about 20 cm to 25 cm and a circumference of about 15 cm to 30 cm. In another example, a wearable device adapted for use on a thigh of a user 102 may have a length of about 30 cm and a circumference of about 40 cm to 60 cm. In another example, a length of a device adapted to be used for an upper and lower arm may have a length of about 40 cm to 60 cm. In another example, a length of a device adapted to be used for an upper and lower arm may have a length of about 60 cm to 80 cm. A number and spacing of flexible elements may be similarly adapted for use with different limbs or portions of limbs. With reference to FIGS. 2A and 2B an example of a peristaltic pressure wave 108 induced by a wearable device 100 is shown. The wearable device 100 can induce at least one peristaltic pressure wave 108, e.g., a first and a second peristaltic pressure wave 108. In the example shown, the pressure wave 108 is a cranial/proximal pressure wave 108 (e.g., a wave that tends to move fluid toward the heart of the user 102). As shown for example, the pressure wave 108 begins low on the user's 102 limb 106, constricting a portion of the limb 106. The pressure wave 108 travels up the limb 106 as the wearable device 100 progressively constricts the limb 106 further up toward the user's 102 heart, while releasing the lower portions of the limb 106. The pressure wave 108 may reset once it reaches an end (e.g., top or bottom) of the wearable device 100. For example, when the 108 reaches the top of the pressure wave 108, a new pressure wave 108 may be initiated by the wearable device 100 at the lower end of the limb 106. In some embodiments, multiple pressure waves 108 may travel along the wearable device 100 at the same time. A wearable device 100 may be configured to apply either a cranial/proximal pressure wave 108 or a caudal/distal pressure wave 108, depending on the user's preference or therapeutic needs. In some embodiments, the direction of a pressure wave 108 can be changed between cranial/proximal and caudal/distal by changing a setting or configuration of the wearable device 100.

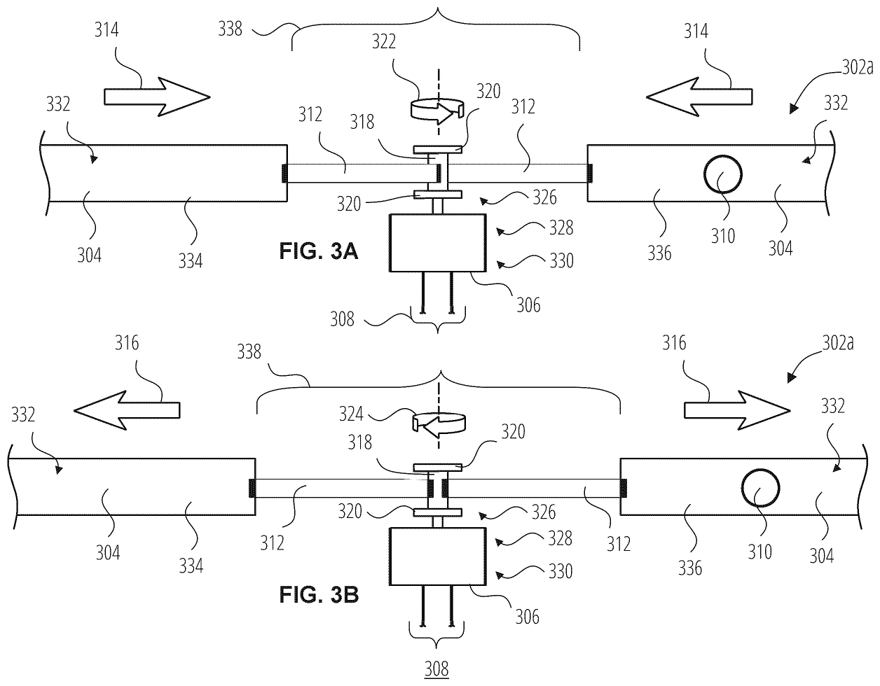

With reference to FIG. 3A-FIG. 3B, and example of an actuator assembly 302a is shown. The actuator assembly 302a includes an element 304 adapted to be worn on a limb 106 of a user 102 and an actuator device 306 (also referred to herein as an actuator 606) adapted to selectively retract and extend an element 304 to provide peristaltic compression of the user's 102 limb 106. In the example, shown, the actuator assembly 302a includes a tension element 312 linked to the element 304 and to a tensioner device 326. The tensioner device 326 selectively retracts the tension elements 312 to retract the element 304. In other embodiments, the tension element 312 may be omitted and the element 304 may be linked directly to the tensioner device 326. In the example shown, an optional sensor 310 is coupled to, disposed on, or integrated with, the element 304.

The element 304 is suitable to be worn on a limb 106 (e.g., forearm, upper arm, calf, thigh, etc.) of a user 102. The element 304 is formed of a body portion 332, with an end portion 334 and an end portion 336. The end portion 334 and the end portion 336 may extend from a tip of the body portion 332 to the other of the end portion 334 or end portion 336. For example, the end portion 334 may form up to about one half of the body portion 332 and the end portion 336 may form up to about the other half of the body portion 332. The body portion 332 may be configured to wrap at least partly around an outer periphery of the limb 106 of the user 102. When so positioned on the limb 106, the end portion 334 and the end portion 336 may be opposed to and facing one another. There may be a gap or distance 338 between the end portion 334 and the end portion 336 when disposed on the limb 106. For example, the end portion 334 and the end portion 336 may define the distance 338 when disposed on the limb 106. The tensioner device 326 may be disposed between the end portion 336 and the end portion 334 in the gap 338.

The element 304 may be a flexible strap with a thin thickness that can be worn on the limb 106 of the user 102 beneath the user's 102 clothes. In some embodiments, the element 304 is relatively inelastic in that it does not appreciably stretch or extend when subjected to a tensile force by the actuator device 306 yet can still bend or fit around the user's 102 limb 106. In some embodiments, the element 304 has an elastic property such that the element 304 stretches when subjected to a tensile force by the actuator device 306 and/or tensioner device 326. The tension element 312 (if used) links the element 304 to the tensioner device 326. In some embodiments, the tension element 312 is flexible and suitable to pull on the element 304. In some embodiments, the tension element 312 is at least partially rigid and can include a structural property such that the element 304 can transmit translational forces such as both pushing and pulling on the element 304. A portion of the tension element 312 may be fixed to the tensioner device 326 such that the tension element 312 can push and/or pull on the element 304 through the action of the tensioner device 326. In various examples, the element 304 and/or tension element 312 may be formed of a woven fabric, a non-woven fabric, a membrane, a strap, etc. The element 304 and/or tension element 312 may be formed of a polymer (e.g., a thermoset or thermoplastic), a metal, natural fibers (e.g., cotton, wool, leather), a composite material, or combinations of these. An elastic element 304 may apply less force to the limb 106 than an inelastic element 304. In some examples, a element 304 may be a cord, string, cable, chain, rubber band, bungee cord, or the like.

The sensor 310 is optional and measures an aspect of the operation of the actuator assembly 302 to measure or control the tension/compression, compression frequency, amount of compression, total time, and/or cycle count that the actuator assembly 302 applies to the limb 106 of the user 102. The sensor 310 is typically, e.g., can be, coupled (e.g., stitched or adhered with an adhesive) to the outer surface of the element 304 (e.g., where element 304 is disposed between the sensor 310 and the limb 106). In some embodiments, the sensor 310 may be integrated with (e.g., sewn or cast) into the element 304. In some embodiments, the sensor 310 may be received between layers of material that form the element 304. In some embodiments, the sensor 310 may be coupled to an interior surface of the element 304, such that the sensor 310 is disposed between the element 304 and the limb 106. The sensor 310 may be similarly disposed on, with, or in the tension element 312. In some embodiments, the sensor 310 is a force sensing resistor, strain gauge, Wheatstone bridge, or the like. In some embodiments, the sensor 310 may be a current or torque sensor integrated with, or coupled to, the actuator device 306 or a drive circuit therefore.

In many embodiments, the actuator device 306 is a rotary actuator suitable to rotate the tensioner device 326. In many embodiments, the actuator device 306 includes a motor 330 (e.g., a stepper motor or servo motor) and a transmission 328 (such as a gear reducer) that adapts torque and/or speed of the raw output of the motor 330 to values suitable to rotate the tensioner device 326 with respect to the limb 106 of the user 102. The actuator device 306 and/or transmission 328 output may be torsionally coupled to the tensioner device 326 such as by a key, spline, set screw, adhesive, integral forming with the tensioner device 326, to transmit torque to and from the actuator device 306 to the tensioner device 326. In some embodiments, the actuator device 306 does not include a transmission 328 such that the motor 330 directly drives the tensioner device 326. In many embodiments, the actuator device 306 is reversible and can be powered in two opposite rotation directions. In many embodiments, one or more conductors 308 such as wires may supply electrical power to the actuator device 306 from a power supply 606. In some embodiments, the actuator device 306 is a pneumatic or hydraulic actuator. In such embodiments, the conductors 308 may be replaced with fluid conduits adapted to receive a flow of a working fluid such as a gas or liquid to power the actuator device 306.

The example tensioner device 326 shown includes a shaft 318 and one or more flanges 320. Different tensioner devices 326 may have respective shafts 318 that may be different diameters than other tensioner devices 326 in the wearable device 100, such as a shaft to allow same amount of rotation of the tensioner device 326 have different compressive effects depending on a specific location along the limb 106. In some embodiments, the shaft 318 may be a cam to have increasing or decreasing impact on the compression relative to the amount of rotation of the shaft 318. The flanges 320 are disposed at opposite end portions of the shaft 318. In some examples, the tensioner device 326 may be in the form of a spool suitable to have portions of the tension element 312 wrapped thereabout as the tensioner device 326 is rotated by the actuator device 306. In some examples, two or more tension elements 312 may be disposed on opposite circumferential surfaces (e.g., front and rear) of the shaft 318. The flanges 320 may guide the tension elements 312 as the tensioner device 326 is turned by the actuator device 306, such as to reduce to prevent the slippage of the tension element 312 from the shaft 318.

The tensioner device 326 may be configured to change (e.g., reduce or increase the size of the gap 338 between the facing end portion 334 and the end portion 336 such as to selectively tension the body portion 332 and compress the limb 106. Regardless of the size of the limb 106, by moving the ends of the strap toward one another, the circumference or diameter of the loop formed by the element 304 is reduced and may compress the user's 102 limb 106. For example, as shown in FIG. 3A, as the actuator device 306 is selectively activated, (e.g., rotated), the tensioner device 326 is rotated by the actuator device 306 with a contraction rotation 322. As the tensioner device 326 rotates, the tension elements 312 (if used) and/or element 304 wrap around the shaft 318 of the tensioner device 326. As the tension element 312/element 304 is wrapped about the shaft 318, the gap 338 effectively shortens in the contraction direction 314. The tensioner device 326 draws the end portion 334 and the end portion 336 toward one another to cause tension in the element 304. Tension induced in the element 304 compresses the user's 102 limb 106.

As shown for example, in FIG. 3B, as the actuator device 306 is selectively deactivated and/or activated in an opposite direction from the contraction rotation 322 (e.g., an extension rotation 324), the tension element 312 and/or element 304 un-wraps from the shaft 318 of the tensioner device 326. The tensioner device 326 allows the end portion 334 and the end portion 336 to move away from one another (e.g., by an elastic property of the element 304 or by forcing the end portion 334 and the end portion 336 apart such as via a rigid or semi-rigid tension element 312), and the gap 338 effectively lengthens in an extension direction 316, thereby releasing tension in the element 304 and decompressing the limb 106. As the tension element 312 and/or actuator device 306 unwraps, the compression in the user's 102 limb 106 is released.

As discussed above, the sensor 310 is adapted to measure a tension/compression that the actuator assembly 302 applies to the limb 106 of the user 102. For example, the actuator device 306 and the sensor 310 may form part of a control loop, such that the actuator device 306 applies a contraction rotation 322 and compresses the limb 106 until a desired level of compression (e.g., 30 mmHg to 200 mmHg) is sensed by the sensor 310. When the desired level of compression is reached, the rotation of the actuator device 306 may be reversed (e.g., an extension rotation 324 is applied) to relax the compression of the limb 106 by the actuator assembly 302.

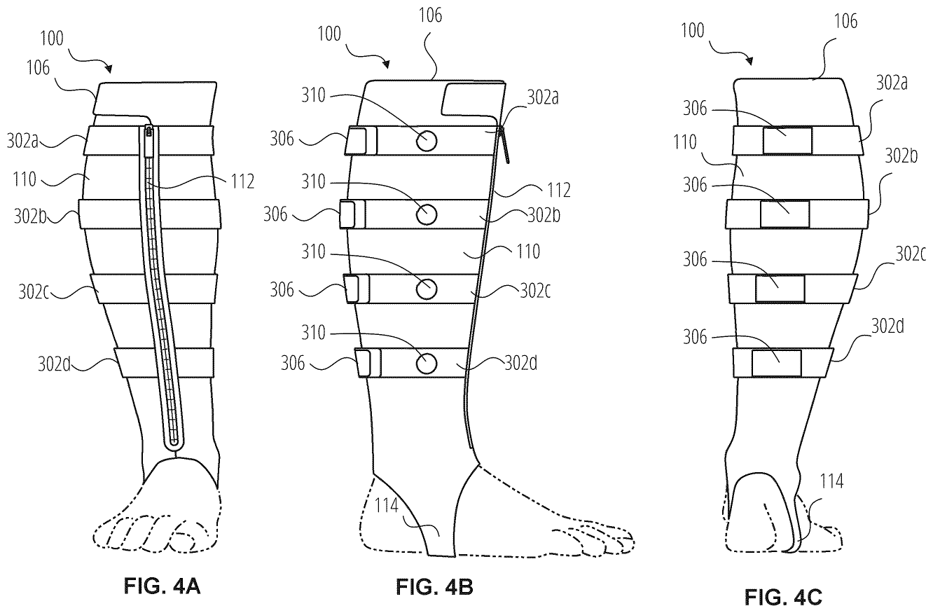

FIGS. 4A-4C show an example of a wearable device 100. The wearable device 100 includes four actuator assemblies 302a-d disposed at intervals along the limb 106 of a user 102. In other examples, more or fewer actuator assemblies 302 may be used.

The actuator assemblies 302a-d in this example are coupled to a sleeve 110 that is selectively wearable on a limb 106 (e.g., calf) of a user 102. The sleeve 110 is openable by a closure mechanism 112 such as a zipper, hook and loop or hook and pile fastener, buttons, clasps, etc. In the example shown, the closure mechanism 112 is disposed on an anterior portion of the limb 106. In other examples, the closure mechanism 112 may be disposed on any other portion of the limb 106, (e.g., lateral, medial, or posterior portion of the sleeve 110) as desired. In some embodiments, the closure mechanism 112 may extend substantially all the way along the length of the sleeve 110 or only partially along the length of the sleeve 110. The sleeve 110 may include a stirrup 114 that fits around a portion of the user's 102 limb (e.g., heel or thumb) to prevent the sleeve 110 from riding up on the limb 106. The sleeve 110 may be formed of a thin woven or non-woven fabric material or membrane. The sleeve 110 may, in many embodiments, have an elastic property such that the sleeve 110 conforms to the shape of the user's 102 limb 106 and may automatically adapt to many different users with limbs of varying lengths and circumferences automatically. The sleeve 110 may include hems or other passages through which the flexible elements 304 may be received.

The actuator assemblies 302a-d may be successively activated or deactivated to form a pressure wave 108 that travels up or down the limb 106 of the user 102. For example, the wearable device 100 may apply a cranial/proximal pressure wave 108 that travels toward the user's head. In another example, the wearable device 100 may apply a caudal/distal pressure wave 108 to the limb 106 that travels away from the user's head. For example, the actuator assembly 302a may be activated thereby constricting the limb 106. The actuator assembly 302b may be activated after the actuator assembly 302a is activated. For example, the actuator assembly 302b may be activated as actuator assembly 302a is being de-activated and its compression of the limb 106 is decreasing. In other examples, the compression from the actuator assembly 302a may be removed from the user's 102 limb 106 before the actuator assembly 302b is activated. When an end actuator assembly 302 (e.g., an actuator assembly disposed at an end portion of the wearable device 100 such as the actuator assemblies 302a or 302d) is activated along the user's 102 limb 106, the pressure wave 108 may begin again by activating an actuator assembly 302 disposed at an opposite end portion of the wearable device 100 (e.g., the other of the actuator assembly 302a or 302d).

In other examples, the respective actuator assemblies 302 may be activated/deactivated in any order and/or duration. By varying the spacing and/or number of actuator assemblies 302a, any number and type of pressure waves 108 may be realized. The speed of the pressure wave 108 may be varied by varying the speed of the actuators 306 and the timing of the activation of successive actuator assemblies 302. The direction of the pressure wave 108 may be reversed by reversing the order of activation of successive actuator assemblies 302. In some examples, two or more simultaneous pressure waves 108 may be applied to the limb 106.

Thus, the wearable device 100 may be easily adapted to a wide variety of body types and limbs (e.g., upper arm, lower arm, calf, foot, or thigh), e.g., because of the sleeve 110 and other components. The wearable device 100 may be adapted to provide either cranial/proximal and/or caudal/distal peristaltic pressure waves 108. The wearable device 100 may include features, elements, and/or benefits of any other wearable device disclosed herein.

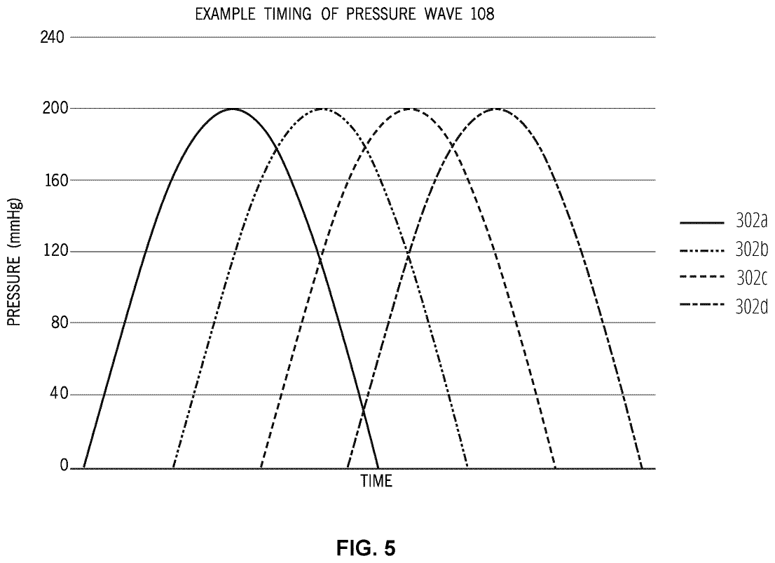

FIG. 5 shows an example of a timing diagram for an example wearable device of the present disclosure, such as the wearable device 100. As shown in FIG. 5, any of the wearable devices disclosed herein may apply a force to the limb of a user in the range of about 0 mmHg to about 200 mmHg. For example, the pressure wave 108 can have a magnitude of between about 30 mmHg and 200 mmHg. In some embodiments, a wearable device 100 disclosed herein may apply a force to a limb of a user in the range of about 0 mmHg to about 30 mmHg. The frequency of contractions may be about 20 to 60 contractions per minute. In some embodiments, the pressure induced in the limb for four actuator assemblies 302a-302d is shown in FIG. 5. As can be seen in FIG. 5, the selective tensioning two actuator assemblies 302 may be temporally spaced from one another such that the wearable device 100 induces a peristaltic pressure wave 108 in the limb. For example, the successive peaks and troughs of the pressure from the actuator assemblies 302a-d causes the peristaltic movement of the pressure wave 108 along the limb of a user thereby moving fluid (lymph, blood, etc.) in a desired direction (e.g., cranial/proximal or caudal/distal). The compressive action of the actuator assemblies 302a may at least partially overlap temporally (see, e.g., the timing of the compression of the actuator assembly 302b, 302c, and 302d overlapping partially with that of the actuator assembly 302a in time, etc.). In the example shown, the compressions of the actuator assemblies 302a-d are substantially sinusoidal in nature. Any other suitable waveform may be used, including but not limited to a square wave, sawtooth, triangle wave, or an irregularly shaped wave.

The calf (gastrocnemius) muscle assists with the pumping action during walking, thereby facilitating venous return, but individuals in career fields that involve prolonged standing or sitting (e.g., doctors, factory workers, office workers, etc.) are at increased risk of a condition known as venous insufficiency, in which the peripheral vascular system becomes overwhelmed by stagnant blood due to inactivation of the calf pump and inability of the passive venous system to return blood to the body's core. The wearable devices disclosed herein may be suitable to treat or prevent venous insufficiency, painful and potentially deadly thrombus, blot clots, lymphatic congestion, lymphedema, and/or edema. For example, the wearable devices, at least in applications for the leg, lower leg and/or calf area of the leg, can help to facilitate the return of blood and other bodily fluids (e.g., lymph or interstitial fluid) by applying sequential external forces that propel fluid from the lower leg back to the heart (FIGS. 1 and 2A/B). The devices and apparatuses are configured to be used on any subject in any environment or occupational scenario where there is a need for the prevention and/or treatment of lower extremity varicose veins, thrombus, and edema, or where a massage device would prove effective. Standing on your feet or sitting for long periods of time can cause thrombus formation, lymphedema, and venous insufficiency as a result of chronic pooling of blood and/or lymph at the feet. Venous insufficiency, in turn, can lead to spider veins and, ultimately, unsightly varicose veins in the lower legs. The current practice of wearing compression socks has limited utility and effectiveness. Surgeons, physicians, nurses, technicians, and others who spend long periods of time standing are at risk of varicose veins or other serious health conditions that may arise like thrombus development or lymphedema. Additionally, spending hours at a time sitting at a desk, which is routine for many working professionals and students, can increase risk of developing venous insufficiency, thrombus or lymphedema. The devices of the present disclosure also move other bodily fluids such as interstitial and lymph fluid toward the center of the body and prevent pooling or clotting.

In applications such as, but not limited to, space exploration, the devices of the present application may treat or prevent orthostatic intolerance by mitigating the risk of postural hypotension (low blood pressure) upon transitioning between two environments with different gravitational forces. For example, following space travel in the weightless environment, hemodynamic changes that often arise during/after landing on another planetary surface can lead to refractory postural hypotension, or orthostatic intolerance. The wearable devices described herein may serve to address these and other, related needs. Further, the wearable devices described herein have pertinent applications in the inflight phase of space travel, during which the body is exposed to microgravity (weightlessness). Hemodynamic changes related to the cephalad fluid shifts observed in this setting may be controlled/mitigated by application of the present wearable device, but with reversal of the peristaltic pumping direction, such that dynamic pressure is applied in the caudal/distal direction. Conditions such as spaceflight-associated neuro-ocular syndrome (SANS) and other, similar physiologic effects of exposure to the spaceflight environment may be controlled and/or mitigated with the application of an embodiment of any wearable device disclosed herein.

Unlike existing products, e.g., compression socks/sleeves, presently disclosed apparatuses and devices do not stretch over time and wear out, and therefore provide a long-term solution. Unlike bulky sequential compression devices used in hospital settings, which are large, noisy (typically pneumatic) machines that prevent users from being ambulatory while using the sequential compression device, the wearable devices of the present disclosure can be worn by users in their normal activities (e.g., walking, sitting, running, standing, etc.) Existing devices, like sequential compression devices, do not induce peristaltic waves but rather bulk-compress a large portion or all of a limb and then release it. Furthermore, such existing devices do not circumferentially surround a limb and move fluid in a desired direction as with devices of the present disclosure.

Provided herein are wearable apparatuses and/or devices for treating venous insufficiency in a variety of settings and applications, and/or for providing massage therapy. The devices and apparatuses are configured to be used on any subject in any environment or occupational scenario where there is a need for the prevention and/or treatment of lower extremity varicose veins and edema, or where a massage device would prove effective. Standing on your feet for long periods of time can cause venous insufficiency as a result of chronic pooling of blood at the feet. This, in turn, can lead to spider veins and, ultimately, unsightly varicose veins in the lower legs. The current practice of wearing compression socks has limited utility and effectiveness.

By way of example and not limitation, professionals in the medical field are in need of such devices and apparatuses, particularly during a pandemic where long hours are required. Surgeons, physicians, nurses, technicians, and others who spend long periods of time standing are at risk of varicose veins.

Additionally, spending hours at a time sitting at a desk, which is routine for many working professionals and students, can increase risk of developing venous insufficiency. Likewise for truck drivers, transport pilots, and others who spend a lot of time sitting.

As another example, the disclosed devices and apparatuses can be configured for applications for bedridden/post-operative/Intensive Care Unit (ICU) patients, where venous return and blood clot prevention are critical.

As another example, the disclosed devices and apparatuses can be configured for use by high-performance jet pilots. The device can be configured with a specific contraction pattern that can replace the current pneumatic G-suit and help to prevent G-induced loss of consciousness on maneuvering.

As another example, the disclosed devices and apparatuses can be configured for applications in space flight and use by astronauts. Astronauts often suffer from the exact opposite problem from that described above: venous return is highly effective in microgravity, and both blood and extravascular fluid tend to stagnate in the head, neck, and upper torso, causing a litany of problems affecting vision, olfaction, and overall cardiopulmonary function. Such an application can help decrease the fluid shifts seen following adaptation to the spaceflight environment. More particularly, a modified version of the disclosed device and apparatus can be configured to pump in the opposite direction, toward the feet, to reduce headward fluid shifts and associated harmful physiologic processes by reversing pump direction and decreasing venous return during space flight.

As a final non-limiting example the disclosed devices and apparatuses can be used by athletes to reduce workout recovery time and maximize training value by enhancing post-workout blood flow, including by providing massage therapy.

Taken together, in some embodiments, peristaltic movement can be cranial/proximal for some applications (e.g., terrestrial/venous/lymphatic), and caudal/distal for other applications (e.g., spaceflight use). In some embodiments, the disclosed devices can be configured such that a user or practitioner can select between cranial/proximal and caudal/distal peristaltic movement.

In addition to treating venous insufficiency, some embodiments of the disclosed devices and apparatuses can be configured to fit the upper extremity and may provide a tremendously effective mechanism for reducing lymphedema associated arm swelling after surgical treatments for conditions such as breast cancer: by propelling lymph flow back toward the torso, lymphatic congestion can be eliminated.

These and other applications, as would be appreciated by one of ordinary skill in the art, are provided, particularly where there is a need to treat and/or prevent venous insufficiency, lymphedema release, blood clot relief, extravascular fluids, i.e. lymph and the like.

The presently disclosed apparatuses and devices make up for absent gastrocnemius contraction by applying pressure in peristaltic waves to keep blood from pooling in extremities and appendages, including for example the lower legs. Unlike existing products, e.g., compression socks/sleeves, presently disclosed apparatuses and devices do not stretch over time and wear out, and therefor provide a long-term solution. Additionally, the presently disclosed apparatuses and devices are easy to use, hassle-free, and consumer friendly, even in strict environments such as medical facilities, operating rooms, etc. Moreover, the presently disclosed apparatuses and devices can in some embodiments be reasonably quiet and/or inaudible such that during use the devices do not cause a distraction or otherwise interfere with the user and others around the user.

It will be understood that various details of the presently disclosed subject matter may be changed without departing from the scope of the presently disclosed subject matter. Furthermore, the foregoing description is for the purpose of illustration only, and not for the purpose of limitation.

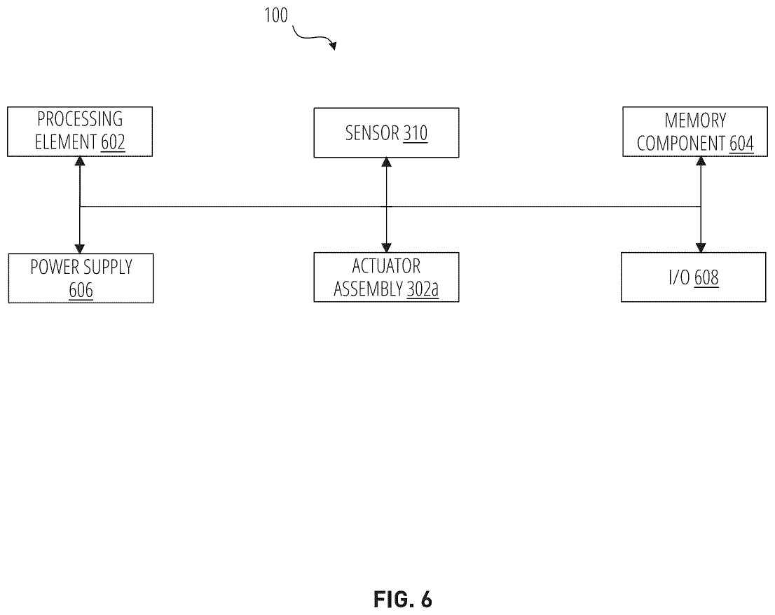

FIG. 6 illustrates a simplified block diagram for the various devices of the wearable device 100. As shown, the various devices may include one or more processing element 602, one or more memory components 604, a sensor 310, a power supply 606, an actuator assembly 302, and an input/output interface 608 (“I/O”), where the various components may be in direct or indirect communication with one another, such as via one or more system buses, contract traces, wiring, or via wireless mechanisms. Certain devices such as the memory component 604, the processing element 602, the sensor 310, and the input/output interface 608 may be optional

The one or more processing element 602 may be substantially any electronic device capable of processing, receiving, and/or transmitting instructions. For example, the processing element 602 may be a microprocessor, microcomputer, graphics processing unit, or the like. It also should be noted that the processing element 602 may include one or more processing elements or modules that may or may not be in communication with one another. For example, a first processing element may control a first set of components of the wearable device 100 and a second processing element may control a second set of components of the wearable device 100 where the first and second processing elements may or may not be in communication with each other. Relatedly, the processing elements may be configured to execute one or more instructions in parallel locally, and/or across a network, such as through cloud computing resources.

The memory component 604 stores electronic data that may be utilized by the wearable device 100, such as audio files, video files, document files, programming instructions, and the like. The memory component 604 may be, for example, non-volatile storage, a magnetic storage medium, optical storage medium, magneto-optical storage medium, read only memory, random access memory, erasable programmable memory, flash memory, or a combination of one or more types of memory components.

The wearable device 100 may also include a power supply 606. The power supply 606 provides power to various components of the wearable device 100. The power supply 606 may include one or more rechargeable, disposable, or hardwire sources, e.g., batteries, power cord, AC/DC inverter, DC/DC converter, or the like. Additionally, the power supply 606 may include one or more types of connectors or components that provide different types of power to the wearable device 100. In some embodiments, the power supply 606 may include a connector (such as a universal serial bus) that provides power to the computer or batteries within the computer and also transmits data to and from the device to other devices. As previously discussed, the wearable device 100 may include a portable power supply such as a battery to power the actuator assemblies 302. In some embodiments, the wearable device 100 may be powered by a corded power supply.

The optional input/output interface 608 allows the wearable device 100 to receive input from a user and provide output to a user, e.g., the user 102. For example, the input/output interface 608 may include a capacitive touch screen, keyboard, mouse, stylus, or the like. The type of devices that interact via the input/output interface 608 may be varied as desired. The input/output interface 608 may include a network interface that receives and transmits data to and from a network. The network interface may transmit and send data to the network directly or indirectly. For example, the networking/communication interface may transmit data to and from computing devices through the network. In some embodiments, the network interface may also include various modules, such as an application program interface (API) that interfaces and translates requests across the network to a server or other computing device. The network interface may be any suitable wired or wireless interface. For example, the network may be an Ethernet network, Wi-Fi, Bluetooth, Wi-Max, Zigbee network, the internet, microwave link, or the like.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. An apparatus for treating venous deficiency comprising:
a plurality of actuator assemblies positioned along a limb of a user, each of the plurality of actuator assemblies including:
a flexible element including a body portion and two opposing end portions, wherein the body portion is configured to at least partly extend around an outer periphery of the limb;
an actuator coupled to the opposing end portions and configured to, via a tensioner device, selectively reduce a distance between the two end portions to compress the limb.

2. The apparatus of claim 1, wherein the plurality of actuator assemblies comprises a first actuator assembly and a second actuator assembly, and the selective reduction of the distance of the first actuator assembly is temporally spaced from the selective reduction of distance of the second actuator assembly such that the apparatus induces a peristaltic pressure wave in the limb.

3. The apparatus of claim 1, wherein the actuator comprises a rotary actuator.

4. The apparatus of claim 3, wherein the rotary actuator comprises one of the at least one of a stepper motor or a servo motor.

5. The apparatus of claim 3, wherein the tensioner device is rotationally coupled to the rotary actuator and to the opposing end portions, such that a rotation of the rotary actuator causes the end portions of the flexible element to at least partially wrap around the tensioner device to reduce the distance between the two end portions.

6. The apparatus of claim 1, further comprising a sleeve wearable on the limb of the user, wherein the plurality of actuator assemblies is coupled to the sleeve.

7. The apparatus of claim 6, further comprising a closure mechanism coupled to the sleeve and operative to selectively open the sleeve for ease of placement on the limb.

8. The apparatus of claim 7, wherein the closure mechanism comprises at least one of a zipper, a button, a clasp, a hook and loop fastener, or a hook and pile fastener.

9. The apparatus of claim 1, further comprising a sensor configured to measure one or more of a tension in the flexible element or a compression in the limb.

10. The apparatus of claim 9, wherein the apparatus tensions the flexible element until a desired level of compression in the limb is applied by the flexible element, as measured by the sensor.

11. The apparatus of claim 10, wherein the desired level of compression ranges from about 30 mmHg to 200 mmHg.

12. The apparatus of claim 2, wherein the selective reduction of the distance of the first actuator assembly and the selective reduction of distance of the second actuator assembly at least partially overlap temporally.

13. The apparatus of claim 9, wherein the sensor comprises one or more of a force sensing resistor, a strain gauge, or a Wheatstone bridge.

14. The apparatus of claim 9, wherein the sensor is coupled to the flexible element.

15. The apparatus of claim 14, wherein the sensor is coupled to the flexible element by one or more of stitching or adhesive.

16. The apparatus of claim 9, wherein:
the flexible element comprises a plurality of layers; and
the sensor is disposed between at least two layers of the plurality of layers.

17. The apparatus of claim 2, wherein an order of the selective reduction of distance of the first actuator assembly and the selective reduction of distance of the second actuator assembly is such that the peristaltic pressure wave is a proximal pressure wave.

18. The apparatus of claim 2, wherein an order of the selective reduction of distance of the first actuator assembly and the selective reduction of distance of the second actuator assembly is such that the peristaltic pressure wave is a distal pressure wave.

19. The apparatus of claim 6, wherein the sleeve comprises a woven or non-woven fabric.

20. The apparatus of claim 6, wherein the sleeve is conformable to the limb.

21. The apparatus of claim 6, wherein the sleeve has a circumference of about 10 cm to about 60 cm.

22. The apparatus of claim 6, wherein the sleeve has a thickness of about 5 mm to about 1.5 cm.

23. The apparatus of claim 2, wherein the peristaltic pressure wave has a frequency of about 20 to 60 waves per minute.

24. The apparatus of claim 2, wherein the apparatus induces a second peristaltic pressure wave in the limb at the same time and different location as the peristaltic pressure wave.

25. A method of treating venous deficiency comprising:
applying a wearable device to a limb of a user, wherein the wearable device comprises:
a plurality of actuator assemblies receivable on the limb of the user, each of the plurality of actuator assemblies including:
a flexible element including a body portion and two opposing end portions, wherein the body portion is configured to at least partly extend around an outer periphery of the limb; and
an actuator coupled to the opposing end portions and configured to selectively reduce a distance between the two end portions to compress the limb, wherein the selective reduction of distance of adjacent actuator assemblies of the plurality of actuator assemblies induce a peristaltic pressure wave in the limb.

26. The method of claim 25, wherein the peristaltic pressure wave has a magnitude of between about 30 mmHg and 200 mmHg.

27. The method of claim 25, wherein the peristaltic pressure wave has a frequency of about 20 to 60 waves per minute.
